(19) United States
(12) Patent Application Publication
(10) Pub. No.: US 2021/0310080 A1
(43) Pub. Date: Oct. 7, 2021

(54) Composition for diagnosing cancer using potassium channel proteins

(71) Applicant: Cellionbiomed Inc., Sejong-si (KR)

(72) Inventors: Suk Hyo SUH, Goyang-si (KR); Shin Kyu CHOI, Seoul (KR); Ji Aee KIM, Seoul (KR)

(21) Appl. No.: 17/354,008

(22) Filed: Jun. 22, 2021

Related U.S. Application Data
(62) Division of application No. 16/307,972, filed on Jan. 17, 2019, now abandoned.

(30) Foreign Application Priority Data
Jun. 20, 2016 (KR) 10-2016-0076767

(51) Int. Cl. C12Q 1/6886; G01N 33/574

(52) U.S. Cl. CPC C12Q 1/6886; G01N 33/57496; G01N 33/574; G01N 2800/54; C12Q 2600/158; C12Q 2600/118; G01N 33/57438

(57) ABSTRACT

This disclosure relates to a composition for diagnosing cancer by using potassium channel proteins; to a kit for diagnosing cancer comprising the composition; and to an information providing method for diagnosing cancer. Specifically, the composition or kit for diagnosing cancer provided in this disclosure may be used to diagnose the onset of cancer regardless of its type, by measuring the expression levels of potassium channels, KCa3.1 channel and KCa2.3 channel, or a regulator thereof from vascular endothelial cells treated with a sample of a subject, or from red blood cells isolated from the subject, and thus can be widely utilized in determining the stages of progression (growth, metastasis, prognosis, and recurrence) of various cancers.

### BACKGROUND

### 1. Field

This disclosure relates to a composition for diagnosing cancer using potassium channel proteins, a kit for diagnosing cancer including the composition, and a method for providing information for cancer diagnosis.

### 2. Description of Related Art

‘Tumors’ are divided into benign tumors and malignant tumors. Benign tumors are slow to grow and do not metastasize. On the other hand, malignant tumors, often called cancer, rapidly grow while invading nearby tissues and become life-threatening by being metastasized to other organs. According to statistics in Korea, cancer deaths are the most common cause which accounted for 28.3% of all deaths in 2013, and the number of cancer patients is increasing every year, like more than 200,000 new cancer patients each year. The probability of getting cancer is very high, reaching 36.2% during his/her lifetime of 81 years, which is close to the average life expectancy of Korean people. Cancer, the disease that has the greatest impact on human health, is diagnosed by histologic detection of a cancer mass using radiological methods such as endoscopy and computed tomography (CT). These traditional methods are discoverable only after the cancer mass has reached a detectable size. That is, the cancer has progressed to some extent. However, finding and treating cancer properly at an early stage is important. Therefore, many methods for early detection of cancer development or recurrence are being studied.

A great deal of development research is currently under way on use of specific cancer markers such as tumor markers for early detection of cancer. The tumor markers that are used are carcinoembryonic antigen (CEA) for colon cancer and a-fetoprotein (AFP) for liver cancer. Korean Patent Publication No. 10-2009-0029868 discloses a hepatocellular carcinoma diagnostic method using a tumor associated marker of hepatocellular carcinoma in human serum to enhance the accuracy of hepatocellular carcinoma diagnosis by using overexpression phenomenon of the annexin 2. KR Patent Registration No, 10-1058783 discloses a hepatocellular carcinoma diagnostic method using a cyclophilin A-encoding gene as a marker for liver cancer diagnosis. KR Patent Registration No. 10-1071219 discloses a hepatocellular carcinoma diagnostic method using a polymorphic mark for the diagnosis of hepatocellular carcinoma based on polymorphisms present in exons of the TGFβR III gene. These cancer diagnostic methods using such tumor markers are being used or are being developed as auxiliary diagnostic methods for early diagnosis of cancer.

The inventors have found that expression levels of KCa2.3 and KCa3.1 proteins are significantly increased in liver cancer, lung cancer, and pancreatic cancer. The increase in expression levels of KCa2.3 and KCa3.1 proteins is caused by vascular growth factors such as VEGF secreted by cancer cells for cancer tissue growth. It is found that this increase in the expression of K+ channel proteins occurs very rapidly since it does occurs within 24 hours or less even when normal vascular endothelial cells are exposed to patient serums. It is also found that expression of regulatory factors (clathrin and the like) that regulate the expression of K+channel proteins is also very rapidly regulated. These results suggest that the expression levels of the KCa2.3 and KCa3.1 proteins and regulatory factors of these K+ channel proteins reflect the level of vascular growth factors. KCa3.1 protein expression is also increased in the patient's red blood cells. Since vascular endothelial cells and red blood cells are exposed to the same serum, the expression level of KCa3.1 in vascular endothelial cells may be replaced with that in red blood cells.

Under these circumstances, the present inventors have made intensive researches to develop a method for effectively diagnosing cancer through measurement of expression levels of angiogenesis-related factors. As a result, it has been found that onset of cancer can be diagnosed in early stage by measuring the expression levels of potassium channels, KCa3.1 channel and KCa2.3 channel, or a regulatory factor thereof from vascular endothelial cells or from red blood cells.

### SUMMARY

An object of this disclosure is to provide a composition for diagnosing cancer.

Another object of this disclosure is to provide a kit for diagnosing cancer comprising the composition.

Still another object of this disclosure is to provide a method for providing information for cancer diagnosis.

In one general aspect, there is provided a composition for diagnosing cancer including: a formulation capable of measuring an expression level of the mRNA expressed from a potassium channel protein or a gene encoding the protein. Here, the potassium channel protein may be a Kca2.3 channel, a KCa3.1 channel or a combination thereof.

Since an expression level of a potassium channel (Kca2.3 channel or KCa3.1 channel) is increased in normal vascular endothelial cells treated with serum from a cancer patient or red blood cells of a cancer patient, the composition including a formulation capable of measuring the channel may be used as follows to diagnose cancer by measuring expression levels of the potassium channel (Kca2.3 channel or KCa3.1 channel) in the vascular endothelial cell or the red blood cells. In other words, since it is known that the expression level of KCa2.3 channel or KCa3.1 channel affects cancer metastasis and prognosis, the prognosis and the probability of metastasis may be determined depending on the expression degree of these channels. When the expression of these channels, which has been decreased after cancer treatment, increases, the expression of VEGF secreted from recurrent cancer cells may be increased, which can be thus used for the diagnosis of cancer recurrence.

Furthermore, the composition for diagnosis of this disclosure can be widely utilized in determining the stages of progression (growth, metastasis, prognosis, and recurrence) of various cancers since the expression is increased during progression from hepatitis to liver cirrhosis or from liver cirrhosis to liver cancer.

The technique for diagnosing cancer by measuring the expression level of potassium channel (Kca2.3 channel or KCa3.1 channel) or a regulatory factor thereof from vascular endothelial cells treated with a blood sample of a subject or red blood cells isolated from a subject has never been known until now and first developed by the inventors.

The technique for diagnosing cancer provided by this disclosure is expected to be useful for early diagnosis to determine the probability of recurrence after cancer treatment. Researches of early diagnosis to determine the probability of recurrence after cancer treatment are under way because many cancer patients have recurrences and the cure rate is high when it is caught and treated early before cancer cells spread. Recently, the British Cancer Institute has developed a method to detect the probability of recurrence by detecting the DNA released from breast cancer cells before blood cancer cells invade into other tissues. This method is expected to diagnose recurrence of cancer several months earlier before an existing method such as CT, MRI or the like can detect recurrent cancer mass. It may be useful for the diagnosis to predict cancer recurrence because secretion of angiogenesis factor and increases in the expression of KCa2.3 and KCa3.1 thereby may occur before cancer recurrence, that is, before a cancer mass grows. It may also be expected to be very useful for predicting the probability of metastasis and prognosis of cancer. It is reported that increase in the expression of KCa3.1 increases the likelihood of metastasis and reduces the likelihood of survival in some cancers. It is also reported that the prognosis is bad when vascular endothelial growth factor (VEGF) receptors, which increase the expression of KCa2.3 and KCa3.1, are increased in liver cancer. Determining the KCa2.3 and KCa3.1 expression levels may be thus useful to predict the probability of cancer metastasis and prognosis.

The cancer that can be diagnosed using the composition may not be particularly limited as long as levels of the protein or mRNA of KCa3.1 channel or KCa2.3 channel in vascular endothelial cells or red blood cells is increased due to the onset of cancer. Examples of the cancer may include liver cancer, lung cancer, gastric cancer, pancreatic cancer, renal cell carcinoma, uterine cancer, cervical cancer, brain cancer, oral cancer, colon cancer, biliary cancer, bone cancer, skin cancer and the like. It may be caused alone or in combination.

As used herein, the term “potassium channel protein” refers to a channel protein present in the membrane and allowing the flow of K⁺ ions among ion channel proteins, which are membrane proteins allowing the flow of ions from one side of the membrane to the other. In general, ion channel proteins are classified into a Na⁺ channel, a Ca²⁺ channel and a K⁺ channel depending on the type of ions that pass through. Among them, potassium channel proteins regulate the flow of ions through the cell membrane to determine a membrane voltage. Thus, the potassium channel proteins have a great effect on cell functions such as controlling intracellular Ca²⁺ concentration and membrane excitability. It is known that there are several kinds of K⁺ channels, which can be activated by intracellular Ca²⁺ concentration, including a KCa1.1 channel, a KCa2.3 channel, a KCa3.1 channel and the like in vascular endothelial cells.

In this disclosure, the potassium channel protein is not particularly limited, but a KCa3.1 channel protein or a KCa2.3 channel protein may be used alone or in combination. Particularly, the potassium channel may be KCa3.1 or KCa2.3 for vascular endothelial cells and KCa3.1 for red blood cells, but is not limited thereto.

As used herein, the term “KCa3.1 channel (intermediate conductance calcium-activated potassium channel, subfamily N, member 4)” refers to a heterotetrameric voltage-independent potassium channel protein that is expressed from the KCNN4 gene and is activated by intracellular calcium. The vascular endothelial cell KCa3.1 channel induces hyperpolarization. When hyperpolarization is induced, intracellular Ca²⁺ influx is increased, thereby activating NO formation by eNOS and relaxing blood vessels. In addition, the KCa3.1 channel may induce angiogenesis by promoting the proliferation of vascular endothelial cells. Sequence information of the KCa3.1 channel may be obtained from a known database such as the National Center for Biotechnology Information (NCBI). For example, the KCa3.1 channel of this disclosure may be NCBI GenBank Accession NO. NM_002250, NM_001163510, NP_002241 or NP_001156982, but is not limited thereto.

As used herein, the term “KCa2.3 channel” is referred to as SK3 (small conductance calcium-activated potassium channel 3) and refers to a potassium channel protein expressed from KCNN3 gene. Like the KCa3.1 channel, the vascular endothelial cell KCa2.3 channel may induce hyperpolarization and promote NO formation by eNOS, thereby relaxing blood vessels, promoting the proliferation of the vascular endothelial cells, and inducing angiogenesis. Sequence information of the KCa2.3 channel may be obtained from a known database such as the National Center for Biotechnology Information (NCBI). For example, the KCa2.3 channel of this disclosure may be NCBI GenBank Accession NO. NM_001204087, NM_080466, NP_001191016 or NP_536714, but is not limited thereto.

The composition of this disclosure may further include a formulation capable of measuring an expression level of the mRNA expressed from a protein, which is a regulatory factor of the potassium channel such as clathrin, caveolin1, EEA, Rab5C or the like, or a gene encoding the protein.

When the serum of a cancer patient is treated with vascular endothelial cells, an expression level of the regulatory factor of the potassium channel such as clathrin, caveolin1, EEA, Rab5C or the like in the vascular endothelial cells is decreased or an expression level of the red blood cells isolated from a cancer patient is decreased. The expression level of the regulatory factor may be thus compared with that measured in a normal control group to diagnose cancer. The formulation capable of measuring the protein or mRNA level of the regulatory factor of the potassium channel may be added to the composition for diagnosing cancer so that the accuracy of cancer diagnosis is improved.

As used herein, the term “caveolin1” is a major component of the caveolae plasma membrane found in most cell types and is associated with an initiating step in coupling integrins to the Ras-ERK pathway and a cell cycle progression. Sequence information of the caveolin1 may be obtained from a known database such as the National Center for Biotechnology Information (NCBI). For example, the caveolin1 of this disclosure may be NCBI GenBank Accession NO. NM_001172897.1, NM_001243064.1, NM_031556.3 or NM_001135818.1, but is not limited thereto.

As used herein, the term “clathrin” is a protein that plays a role in the formation of coated vesicles and forms a triskelion shape composed of three clathrin heavy chains and three light chains. The triskelia interacts to form a polyhedral lattice that surrounds the vesicle. Sequence information of the clathrin may be obtained from a known database such as the National Center for Biotechnology Information (NCBI). For example, the clathrin of this disclosure may be NCBI GenBank Accession NO. NM_001288653.1, NM_001003908.1, NM_019299.1 or XM_001136053.4, but is not limited thereto.

As used herein, the term “Rab5C” is a protein as one of GTPases that controls the fusion of early endosomes and plasma membrane to regulate membrane traffic. Sequence information of the Rab5C may be obtained from a known database such as the National Center for Biotechnology Information (NCBI). For example, the Rab5C of this disclosure may be NCBI GenBank Accession NO.CR541901.1, AB232595.1, NM_001105840.2 or NM_001246383.1, but is not limited thereto.

As used herein, the term “Early Endosome Antigen 1 (EEA1)” localizes to early endosomes and has an important role in endosomal trafficking. Sequence information of the EEA1 may be obtained from a known database such as the National Center for Biotechnology Information (NCBI). For example, the EEA1 of this disclosure may be NCBI GenBank Accession NO. NM_003566.3, NM_001001932.3, NM_001108086.1 or XM_522610.5, but is not limited thereto.

As used herein, the term “formulation capable of measuring an expression level of protein” refers to a formulation capable of specifically binding to a desired protein and measuring its level easily. When such a formulation is used, the level of a target protein may be easily and accurately measured.

The formulation capable of measuring an expression level of protein means a formulation that can be used to measure the level of protein of KCa3.1 channel, KCa2.3 channel or a regulatory factor thereof expressed in vascular endothelial cells or red blood cells. For example, the formulation may be an antibody or an aptamer that can be specifically binding to a target protein to be used for western blotting, enzyme linked immunosorbent assay (ELISA), radioimmunoassay(RIA), radioimmunodiffusion, ouchterlony immunodiffusion, rocket immunoelectrophoresis, immunohistochemistry, immunoprecipitation assay, complement fixation assay, FACS and protein chip assay, or the like.

As used herein, the term “antibody” is a proteinaceous molecule capable of specifically binding to an antigenic site of a protein or peptide molecule. Such an antibody may be prepared by cloning each gene into an expression vector according to a conventional method to obtain a protein encoded by the marker gene, and producing from the obtained protein by a conventional method. The structure of the antibody may not be particularly limited, and polyclonal antibodies, monoclonal antibodies, antigen-binding antibodies or a part thereof may be included in the antibody of this disclosure. The antibody may also include specific antibodies such as humanized antibodies in addition to all immunoglobulin antibodies. The antibody also includes a functional fragment of an antibody molecule as well as a complete form having two full-length light chains and two full-length heavy chains. The functional fragment of an antibody molecule means a fragment having at least an antigen-binding fragment and examples thereof may include Fab, F(ab'), F(ab') 2, Fv, and the like.

The antibody of this disclosure may an antibody capable of specifically binding to a KCa3.1 channel, a KCa2.3 channel or a regulatory factor thereof and examples thereof may include a polyclonal antibody, a monoclonal antibody and a part thereof capable of specifically binding to a KCa3.1 channel, a KCa2.3 channel or a regulatory factor thereof.

As used herein, the term “aptamer” refers to a single-stranded nucleic acid having stable three-dimensional structure (DNA, RNA, or modified nucleic acid) that is capable of binding to a specific target molecule to be detected in a sample. The presence of the target molecule in a sample may be confirmed particularly through the binding. The aptamer may be prepared by preparing a sequence of an oligonucleotide having a selectivity and high affinity to a target protein to be identified according to a general method of preparing an aptamer and then synthesizing an oligonucleotide having —SH, —COOH, —OH or —NH₂ group on 5′-end or 3′-end thereof so as to be able to bind to a functional group of a linker. The aptamer of this disclosure may be an aptamer capable of specifically binding to a KCa3.1 channel, a KCa2.3 channel, or a regulatory factor thereof. For example, the aptamer may be a DNA aptamer that specifically binds to a KCa3.1 channel, a KCa2.3 channel or a regulatory factor thereof.

As used herein, the term “formulation capable of measuring a level of mRNA” means a formulation used for measuring the level of mRNA transcribed from a target gene in order to confirm the expression of the target gene contained in a sample. Examples of the formulation may include, but are not limited to, a probe, a primer, an antisense oligonucleotide, and the like that specifically bind to a target gene used in methods such as RT-PCR, competitive RT-PCR, real-time RTPCR, RNase protection assay, northern blotting, DNA chip analysis or the like.

As used herein, the term “primer” refers to a short nucleic acid sequence containing a short free 3′ hydroxyl group that forms base pairs with a complementary template strand and serves as a starting point to copy the template strand. The primers may initiate DNA synthesis in the presence of reagents and four different nucleoside triphosphates for polymerization reactions (e.g., DNA polymerase or reverse transcriptase) at appropriate buffer solution and temperature. The PCR conditions, lengths of sense and antisense primers may be modified based on those known in the art.

The primer of this disclosure may be used as a means of detecting a gene of a KCa3.1 channel, a KCa2.3 channel, or a regulatory factor thereof included in cDNA by synthesizing the cDNA from mRNA obtained from red blood cells of a subject suspected of having cancer or vascular endothelial cell treated with a serum sample and amplifying the KCa3.1 channel, the KCa2.3 channel, or the regulatory factor thereof contained in the cDNA. A polynucleotide sequence of the primer may not be particularly limited as long as it is able to amplify the gene of the KCa3.1 channel, the KCa2.3 channel or the regulatory factor thereof included in the cDNA.

As used herein, the term “probe” means a nucleic acid fragment of RNA or DNA of variable length ranging from a few to hundreds bases long, which can specifically bind to a gene or mRNA. The probe may be provided as an oligonucleotide probe, a single-stranded DNA probe, a double-stranded DNA probe, a RNA probe or the like, or may be labeled with various means for easier detection.

The probe of this disclosure may be used as a means of synthesizing a cDNA from mRNA obtained from red blood cells of a subject suspected of having cancer or vascular endothelial cell treated with a serum sample and detecting a gene of a KCa3.1 channel, a KCa2.3 channel, or a regulatory factor thereof included in the cDNA. A polynucleotide sequence of the probe may not be particularly limited as long as it is able to amplify the gene of the KCa3.1 channel, the KCa2.3 channel or the regulatory factor thereof included in the cDNA.

As used herein, the term “antisense oligonucleotide” refers to a DNA or a RNA or a derivative thereof including a nucleic acid sequence complementary to the sequence of a specific mRNA, wherein the antisense oligonucleotide binds to the complementary sequence in the mRNA, and thereby blocks its translation into protein. An antisense oligonucleotide sequence refers to a DNA or RNA sequence that is complementary to and capable of binding to the mRNAs of genes. This may be able to inhibit translation of mRNAs of genes, translocation into cytoplasm, maturation, or an essential activity for all other overall biological functions. The length of the antisense oligonucleotide may be 6 to 100 bases, particularly 8 to 60 bases, and more particularly 10 to 40 bases. The antisense oligonucleotides may be synthesized in vitro by any conventional method and administered in vivo, or may be synthesized in vivo. RNA polymerase I may be used to synthesize an antisense oligonucleotide in vitro. One example for synthesizing antisense RNA in vivo is to allow the antisense RNA to be transcribed using a vector in which the origin of the multiple cloning site (MCS) is in the opposite direction. It is appreciated that the antisense RNA may have a translation stop codon in the sequence to block its translation into the corresponding peptide sequence.

As used herein, the term “diagnosis” means a process of identifying or determining the presence or nature of a disease. The diagnosis of this disclosure may be a diagnosis for predicting the probability of recurrence after cancer treatment, a diagnosis for predicting the probability of cancer metastasis, a prognosis after cancer treatment, or the like.

In another general aspect, there is provided a kit for diagnosing cancer including the composition described above.

The kit of this disclosure may be used to diagnose the onset of cancer by measuring levels of protein or mRNA of a KCa3.1 channel or a KCa2.3 channel expressed from red blood cells isolated from a subject suspected of having cancer or vascular endothelial cells treated with a blood sample thereof, but not limited thereto. The kit may include a primer, a probe or an antibody for measuring levels of protein or mRNA, a composition including one or more other components suitable for diagnosis, a solution, or a device. Examples of the kit may include a reverse transcription polymerase chain reaction (RT-PCR) kit, a DNA chip kit, an enzyme-linked immunosorbent assay (ELISA) kit, a protein chip kit, a rapid kit, Kit and the like.

A kit for measuring an expression level of mRNA of a KCa3.1 channel gene or a KCa2.3 channel gene according to an embodiment may be a kit including elements necessary for performing RT-PCR. The RT-PCR kit may include each primer pair specific to the gene, a test tube or an appropriate container, a reaction buffer (various pHs and magnesium concentrations), a deoxynucleotide (dNTPs), an enzyme such as a Taq-polymerase and a reverse transcriptase, a DNase, a RNAse inhibitor, DEPC-water, sterile water, and/or the like. The kit may further include a primer pair specific to a gene used as a quantitative control.

A kit according to another embodiment may include elements necessary for performing DNA chip analysis. The kit for the DNA chip analysis may include a substrate on which a cDNA corresponding to a gene or a fragment thereof is attached as a probe, a reagent for preparing a fluorescent-labeled probe, a formulation, an enzyme and/or the like. The substrate may include a cDNA corresponding to a quantitative control gene or a fragment thereof.

A kit according to still another embodiment may be a kit for protein chip analysis for measuring the level of a protein expressed from a KCa3.1 channel or a KCa2.3 channel gene. The kit may include, but not limited to, a substrate for immunological detection of an antibody, a suitable buffer solution, a secondary antibody labeled with a chromogenic enzyme or fluorescent substance, a chromogenic substrate, and/or the like. The substrate may be, but is not limited to, a nitrocellulose membrane, a 96-well plate synthesized with a polyvinyl resin, a 96-well plate synthesized from a polystyrene resin, a slide glass made of a glass, or the like. The chromogenic enzyme may be, but is not limited to, a peroxidase or an alkaline phosphatase. The fluorescent substance may be, but is not limited to, fluorescein isothiocyanate (FITC), rhodamine B-isothiocyanate (RITC), or the like. The chromogenic substrate may be, but is not limited to, 2,2′-azino-bis(3-ethylbenzothiazoline-6-sulphonic acid (ABTS), o-phenylenediamine(OPD), 3,3′,5,5′-tetramethylbenzidine (TMB), or the like.

In still another general aspect, there is provided a method for providing information for cancer diagnosis using a biological sample isolated from a subject suspected of having cancer. The method for providing information for cancer diagnosis includes: (a) measuring an expression level of the mRNA expressed from a potassium channel protein or a gene encoding the protein in a biological sample isolated from a subject suspected of having cancer; and (b) comparing the expression level of the protein or mRNA measured in the step (a) with an expression level measured in a normal control sample.

When the expression level measured in the step (a) is higher than that measured in the normal control sample, it may be determined that cancer is likely to occur or cancer has been developed in the subject from whom the biological sample is obtained.

The biological sample is not particularly limited as long as it can be used for measuring the expression level of the mRNA expressed from a potassium channel protein or a gene encoding the protein. For example, the biological sample may be a blood sample or a sample including red blood cells. The method for measuring the expression level of mRNA expressed from the potassium channel protein, the protein or the gene encoding the protein is the same described above.

As used herein, the term “subject” refers to, but is not limited to, human beings suffering from cancer, mammals including mice, domestic animals, and the like, cultured fish, and the like.

The method may further include (c) measuring an expression level of mRNA expressed from at least one protein of clathrin, caveolin1, EEA, and Rab5C, or a gene encoding the protein in the biological sample, and (d) comparing the expression level of the protein or mRNA measured in the step (c) with an expression level measured in a normal control sample. When the expression level measured in the step (c) is lower than that measured in the normal control sample, it may be determined that cancer has been developed in the subject from whom the biological sample is obtained.

The method may further include (c) measuring an expression level of mRNA expressed from at least one protein of clathrin, caveolin1, EEA, and Rab5C, or a gene encoding the protein in the biological sample, and (d′) calculating a ratio of each measured value by dividing the value of the expression level measured in the step (a) by the value of the expression level measured in the step (c) to compare the result ratio with a ratio of the value calculated in the normal control sample. When the ratio the value obtained in the step (d′) is higher than that calculated in the normal control sample, it may be determined that cancer is likely to occur or cancer has been developed in the subject from whom the biological sample is obtained.

According to one embodiment, it is noted that an expression level of a KCa3.1 channel is increased in red blood cells of a patient with liver cancer (FIG. 2a), a level of a KCa3.1 channel is increased in red blood cells of a patient with liver cirrhosis (FIG. 2b), expression levels of clathrin, a regulatory factor of the potassium channel, measured in red blood cells of a patient with liver cancer and a patient with liver cirrhosis, are decreased (FIG. 2c), an expression level of a KCa3.1 channel is increased in red blood cells of a patient with pancreatic cancer, while an expression level of clathrin is decreased (FIG. 3), and ratios between each expression level of the KCa3.1 channel measured in red blood cells of a patient with liver cancer, a patient with liver cirrhosis, and a patient with pancreatic cancer and an expression level of the regulatory factor(clathrin or caveolin1) is significantly higher than a ratio (about 1) measured in the control (FIG. 5a and FIG. 5b).

In still another general aspect, there is provided a method for providing information for cancer diagnosis using vascular endothelial cells treated with a sample isolated from a subject suspected of having cancer.

The method for providing information for cancer diagnosis includes: (a) treating vascular endothelial cells with a sample isolated from a subject suspected of having cancer; (b) measuring an expression level of mRNA expressed from a potassium channel protein or a gene encoding the protein in the endothelial cell treated in the step (a); and (c) comparing the measured expression level of the protein or mRNA to an expression level measured in a normal control sample.

When the expression level measured in the step (b) is higher than that measured in a normal control sample, it may be determined that cancer is likely to occur or cancer has been developed in the subject from whom the biological sample is obtained.

The biological sample is not particularly limited as long as it can be used for measuring the expression level of the mRNA expressed from a potassium channel protein or a gene encoding the protein. For example, the biological sample may be a blood sample or a sample including blood, serum, plasma or the like. The method and subject for measuring the expression level of mRNA expressed from the potassium channel protein, the protein or the gene encoding the protein are the same as described above.

The method may further include (d) measuring an expression level of the mRNA expressed from protein of clathrin, caveolin1, EEA, or Rab5C, or a gene encoding the protein in the vascular endothelial cell treated in the step (a); and (e) comparing the expression level of the protein or mRNA measured in the step (d) with an expression level measured in a normal control sample. When the expression level measured in the step (d) is lower than that measured in a normal control sample, it may be determined that cancer has been developed in the subject from whom the biological sample is obtained.

The method may further include (d) measuring an expression level of the mRNA expressed from protein of clathrin, caveolin1, EEA, or Rab5C, or a gene encoding the protein in the vascular endothelial cell treated in the step (a); and (e′) calculating a ratio of each measured value by dividing the value of the expression level measured in the step (b) by the value of the expression level measured in the step (d) to compare the result ratio with a ratio of the value calculated in the normal control sample. When the ratio of the measured value in the step (e′) is higher than that measured in a normal control sample, it may be determined that cancer is likely to occur or cancer has been developed in the subject from whom the biological sample is obtained.

According to one embodiment, it is noted that expression levels of a KCa3.1 channel and a KCa2.3 channel are increased in serum-treated vascular endothelial cells of a patient with liver cancer (FIG. 1) and expression levels of caveolin-1 and EEA are decreased (FIG. 4).

On the other hand, an expression level of a KCa3.1 channel is significantly increased in red blood cells of a patient with liver cancer (FIG. 2a), compared to that in red blood cells of a normal control sample, an expression level of clathrin is decreased in red blood cells of a patient with liver cancer and a patient with liver cirrhosis, and an expression level of a KCa3.1 channel in red blood cells of a patient with pancreatic cancer is increased (FIG. 3), compared to that in red blood cells of a normal control sample.

In addition, expression levels of a KCa3.1 channel and a KCa2.3 channel are also increased in liver tissues of a liver cancer model mouse (FIG. 6).

Therefore, it is possible not only to diagnose the onset of cancer but also to diagnose the probability of cancer early before the onset of cancer by using the expression levels of the protein of potassium channels or regulatory factors thereof in the vascular endothelial cells treated with a blood sample of a patient suspected of having cancer or the red blood cells isolated from a patient, or each ratio of the expression levels measured.

When the composition or the kit for diagnosing cancer is used, the expression level of potassium channels, KCa3.1 channel and KCa2.3 channel, or a regulatory factor thereof can be measured in sample-treated vascular endothelial cells of a subject or red blood cells isolated from a subject to diagnose the onset of cancer regardless of the type of cancer. Thus, the composition or the kit may be widely used to determine progression levels (growth, metastasis, prognosis and recurrence) of various cancers.

### BRIEF DESCRIPTION OF THE DRAWINGS

FIG. 1 is an image of western blot analysis illustrating the result of comparing expression levels of potassium channels, KCa3.1 channel and KCa2.3 channel, measured in blood sample(serum)-treated vascular endothelial cells of a patient with liver cancer to that of a control group, and a graph illustrating the quantitated results of the expression levels of the potassium channels.

FIG. 2a is an image of western blot analysis illustrating the result of comparing the expression level of the potassium channel, KCa3.1 channel, measured in red blood cells of a patient with liver cancer to that of a control group, and a graph illustrating the quantitated result of the expression level of the potassium channel.

FIG. 2b is an image of western blot analysis illustrating the result of comparing the expression level of the potassium channel, KCa3.1 channel, measured in red blood cells of a patient with liver cirrhosis to that of a control group, and a graph illustrating the quantitated result of the expression level of the potassium channel.

FIG. 2c is an image of western blot analysis illustrating the result of comparing the expression level of a regulatory factor of the potassium channel, clathrin, measured in red blood cells of a patient with liver cancer and a patient with liver cirrhosis, to that of a control group, and a graph illustrating the quantitated result of the expression level of the clathrin.

FIG. 3 is an image of western blot analysis illustrating the result of comparing the expression level of a KCa3.1 channel and a regulatory factor thereof, clathrin, measured in red blood cells of a patient with pancreatic cancer to that of a control group, and graphs illustrating the quantitated results of the expression levels of the KCa3.1 channel and the clathrin.

FIG. 4 is images of western blot analysis illustrating the results of comparing the expression levels of the potassium channels, KCa3.1 channel and KCa2.3 channel, and regulatory factors thereof, caveolin 1 and EEA1, measured in blood sample(serum)-treated vascular endothelial cells of a patient with liver cancer in which the blood sample(serum) of the patient with liver cancer is first diluted.

FIG. 5a is a graph illustrating the results of comparing the expression level ratios of KCa3.1 channel and clathrin measured in red blood cells of a patient with liver cancer and a patient with liver cirrhosis.

FIG. 5b is a graph illustrating the results of comparing the expression level ratio of KCa3.1 channel and clathrin measured in red blood cells of a patient with pancreatic cancer.

FIG. 6 is an image of western blot analysis illustrating the result of comparing the expression levels of the KCa3.1 channel or the KCa2.3 channel expressed in liver tissue cells of a liver cancer model mouse (CerS2), in which a gene encoding ceramide synthase 2 is deleted to induce liver cancer, to that of a control group, and a graph illustrating the quantitated result of the expression level of KCa3.1 channel.

Hereinafter, this disclosure will be described in more detail with reference to examples. However, these examples are for illustrative purposes only, and the scope of this disclosure is not limited to these examples.

### Example 1

### Effect of Blood Samples of a Patient with Liver Cancer on Potassium Channels of Vascular Endothelial Cells

Vascular endothelial cells were treated with a blood sample of a patient with liver cancer to determine whether expression levels of a KCa3.1 channel and a KCa2.3 channel are changed or not.

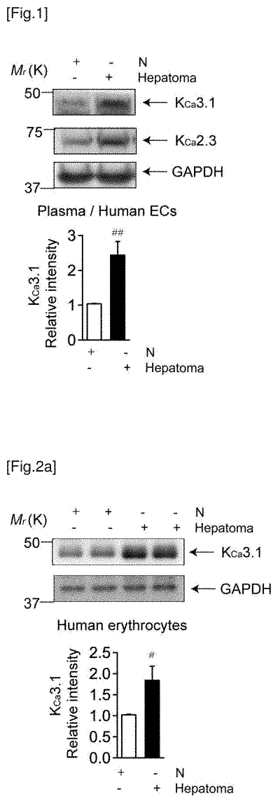

A serum sample was obtained from the blood of a patient with liver cancer. The serum sample was treated with human vascular endothelial cells, and then cultured for 24 hours. After completion of the culture, expression levels of the KCa3.1 channel and the KCa2.3 channel expressed in the vascular endothelial cells were measured by western blot analysis and compared (FIG. 1). Vascular endothelial cells treated with a normal serum sample were used as a control group. The expression level of the KCa3.1 channel protein was measured using an antibody having an amino acid sequence of RQVRLKHRKLREQV (SEQ ID NO: 1), and the expression level of the KCa2.3 channel protein was measured by using an antibody having an amino acid sequence of LHSSPTAFRAPPSSNSTAILHPSSRQGSQLNLNDHLLGHSPSSTA (SEQ ID NO: 2) and particularly binding to the KCa2.3 channel protein. GAPDH was used as an internal control.

As shown in FIG. 1, the expression levels of the KCa3.1 channel and the KCa2.3 channel were increased in the serum sample-treated vascular endothelial cells of the patient with liver cancer, unlike the normal serum sample-treated vascular endothelial cells.

### Example 2

### Analysis of Expression Levels of Potassium Channels and Regulatory Factors Thereof Expressed in Red Blood Cells in a Patient With Liver Cancer and a Patient With Liver Cirrhosis

From the results of Example 1, it was confirmed that the expression levels of the KCa3.1 channel and the KCa2.3 channel were increased in the serum sample-treated vascular endothelial cells of the patient with liver cancer. Therefore, expression levels of potassium channels and regulatory factors thereof were analyzed in red blood cells of the patient with liver cancer.

### Example 2-1

### Analysis pf Expression Levels of Potassium Channels Expressed in Red Blood Cells of a Patient With Liver Cancer and a Patient With Liver Cirrhosis

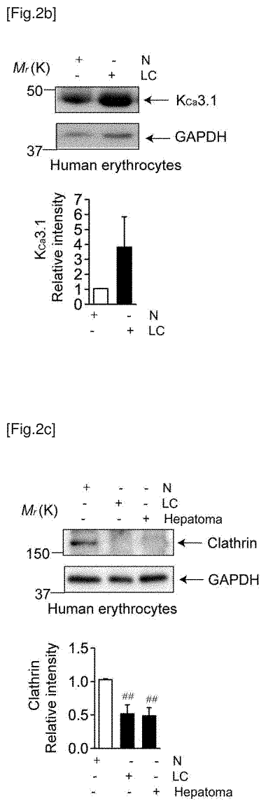

It was confirmed in western blot analysis that the expression level of the KCa3.1 channel was increased in red blood cells of a patient with liver cancer (FIG. 2a). Here, normal human red blood cells were used as a control group and GAPDH was used as an internal control. As shown in FIG. 2a, it was confirmed that the expression of the KCa3.1 channel in the red blood cells of the patient with liver cancer was higher than that of the normal red blood cells.

It was further confirmed in western blot analysis that the expression of the KCa3.1 channel in red blood cells of a patient with liver cirrhosis, instead of the patient with liver cancer, was higher than that of the normal red blood cells (FIG. 2b). Here, normal human red blood cells were used as a control group and GAPDH was used as an internal control.

As shown in FIG. 2b, it was confirmed that the expression of the KCa3.1 channel in the red blood cells of the patient with liver cirrhosis was higher than that of the normal red blood cells.

### Example 2-2

### Analysis of Expression Levels of Regulatory Factors of the Potassium Channel Expressed in Red Blood Cells of a Patient With Liver Cancer and a Patient With Liver Cirrhosis

The expression levels of clathrin, which is known as a regulatory factor of KCa3.1 channel and KCa2.3 channel, in the red blood cells obtained from the blood samples of the patient with liver cancer and the patient with liver cirrhosis used in Example 2-1 were measured by western blot analysis using an antibody having an amino acid sequence of PQLMLTAGPSVAVPPQAPFGYGYTAPPYGQPQPGFGYS (SEQ ID NO: 3) and compared (FIG. 2c). Here, normal human red blood cells were used as a control group and GAPDH was used as an internal control.

As shown in FIG. 2c, it was confirmed that the expression levels of clathrin, a regulatory factor of the potassium channel, were decreased in red blood cells of the patient with liver cancer and the patient with liver cirrhosis in which the expression level of the KCa3.1 channel is increased.

### Example 3

### Analysis of Expression Levels of Potassium Channels and Regulatory Factors Thereof Expressed in Red Blood Cells in a Patient With Pancreatic Cancer

From the results of Example 2, it was confirmed that the expression level of the KCa3.1 channel protein was increased in the red blood cells of the patient with liver cancer and the expression level of the clathrin, the regulatory factor of the KCa3.1 channel protein, was decreased. Thus, it was tested to determine whether the same result would be obtained from a patient with pancreatic cancer.

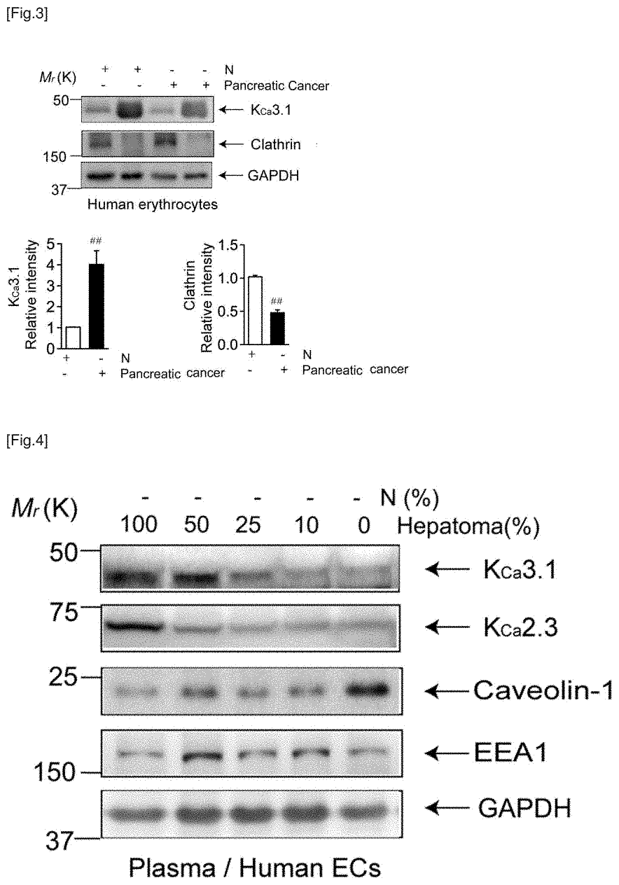

Red blood cells were obtained from the blood of a patient with pancreatic cancer, and then expression levels of the KCa3.1 channel and the clathrin expressed from the red blood cells were measured by western blot analysis and compared (FIG. 3). Here, normal human red blood cells were used as a control group and GAPDH was used as an internal control.

As shown in FIG. 3, it was confirmed that the expression of the KCa3.1 channel in the red blood cells of the patient with pancreatic cancer was increased and the expression of clathrin was decreased as shown in the red blood cells of the patient with liver cancer.

### Example 4

### Effect of Dilution of Blood Samples of a Patient With Liver Cancer on Potassium Channels of Vascular Endothelial Cells

A serum sample was obtained from the blood of a patient with liver cancer. The serum sample was diluted with a culture solution, treated with vascular endothelial cells, and then cultured. After completion of the culture, expression levels of the KCa3.1 channel and the KCa2.3 channel, which are potassium channels, and caveolin1 and EEA1, which are regulatory factors of the potassium channel, expressed in the vascular endothelial cells were measured by western blot analysis and compared. The expression levels of caveolin1 and EEA1 were measured using an antibody having the amino acid sequence of MADELSEKQVYDAHTKEID (SEQ ID NO: 4) and an antibody having the amino acid sequence of FCAECSAKNALTPSSKKPVR (SEQ ID NO: 5), respectively. GAPDH was used as an internal control.

As shown in FIG. 4, the expression levels of the KCa3.1 channel and the KCa2.3 channel were increased in the serum-treated vascular endothelial cells of the patient with liver cancer, but the expression levels of caveolin-1 and EEA1 were decreased at the same time.

### Example 5

### Analysis of Ratios of Expression Levels of Potassium Channels and Regulatory Factors Thereof in Blood Sample-Treated Vascular Endothelial Cells

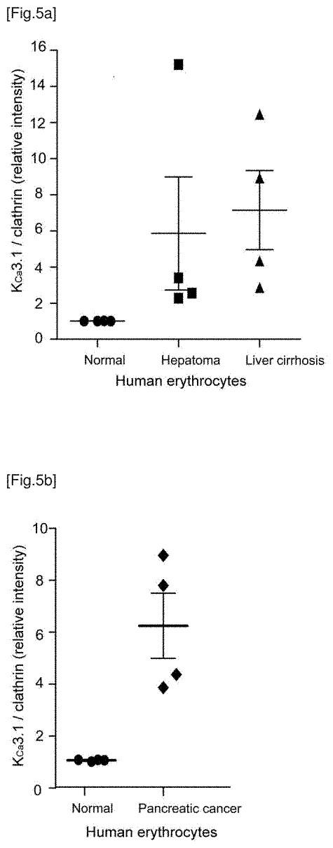

Blood samples (red blood cells or serums) of the patient with liver cancer, the patient with liver cirrhosis and the patient with pancreatic cancer, who were used in Example 1 to Example 4, were treated with vascular endothelial cells and cultured. After completion of the culture, expression levels of the KCa3.1 channel, which is a potassium channel, and clathrin, which is a regulatory factor of the potassium channel, expressed from the vascular endothelial cells were measured. The measured values were applied to the following equation to calculate a ratio of the measured value of the expression level of the channel protein to the measured value of the expression level of the regulatory factor thereof. The calculated ratio was compared to the ratio of the measurements calculated in a normal control (FIG. 5a and FIG. 5b). Here, normal blood sample-treated vascular endothelial cells were used as the control.

### Ratio of Measured Values=Measured Expression Level of KCa3.1 Channel/Measured Expression Level of a Regulatory Factor of the Potassium Channel

As shown in FIG. 5a and FIG. 5b, the calculated ratio of the measured values of the blood sample-treated (red blood cell-treated or serum sample-treated) vascular endothelial cells was 2.0 or higher, while that of the normal control group was about 1.0.

It is noted that when only the expression levels of potassium channels or regulatory factors of the potassium channel are measured and compared and the measured expression levels are similar in a patient with cancer and a normal subject, any error may occur in the diagnosis result. On the other hand, when both the expression levels of potassium channels and regulatory factors of the potassium channel are measured and the ratios thereof are calculated and the measured values are similar, the ratios thereof calculated from a patient with cancer and a normal subject are clearly distinguished from each other so that the likelihood of errors may be significantly reduced in the diagnosis result.

Accordingly, it confirms that the ratio of expression levels between the potassium channels and regulatory factors of the potassium channel may be usefully utilized to determine progression levels of cancers.

### Example 6

### Analysis of Expression Levels of Potassium Channels in Lever Tissue of a Liver Cancer Model Mouse

Since it was confirmed that the expression level of the KCa3.1 channel or the KCa2.3 channel was increased in red blood cells of the patient with liver cancer or the patient with pancreatic cancer in Examples above, the expression level of the potassium channel was measured in a liver tissue of a liver cancer model mouse.

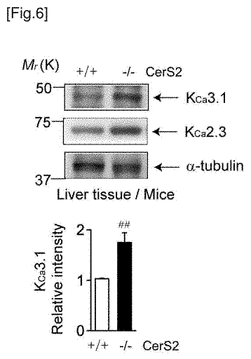

A liver tissue was obtained from a liver cancer model mouse (CerS2) in which the liver cancer was induced by deleting a gene encoding ceramide synthase 2 and expression level of the KCa3.1 channel or the KCa2.3 channel expressed in the obtained liver tissue was measured and quantitated by western blot analysis (FIG. 6). Here, a normal mouse liver tissue was used as a control group and alpha-tubulin was used as an internal control group.

As shown in FIG. 6, it was confirmed that the expression levels of KCa3.1 channel and KCa2.3 channel in the liver tissue of the liver cancer model mouse were increased.

Collectively, the results for Examples described above suggest that cancer patients can be distinguished from normal subjects by utilizing expression levels of the potassium channel proteins in blood sample-treated vascular endothelial cells or red blood cells of cancer patients, expression levels of regulatory factors of the channel proteins, or each ratio of the expression levels.

Similar results were obtained in patients with liver cirrhosis of whom liver cancer was not started but who were more likely to develop liver cancer.

From the above description, it is noted that it is possible not only to diagnose the onset of cancer in patients but also to diagnose the probability of cancer early before the cancer occurs by using the expression level of the protein of the potassium channel or regulatory factors thereof measured in the blood sample-treated vascular endothelial cells of a patient suspected of having cancer or the red blood cells of the patient.

Throughout the description of the present disclosure, when describing a certain technology is determined as that the point of the present disclosure can be fully understood by those who are skilled in the art, the pertinent detailed description has been omitted. While this disclosure includes specific examples, it will be apparent after an understanding of the disclosure of this application that various changes in form and details may be made in these examples without departing from the spirit and scope of the claims and their equivalents. Therefore, the scope of the disclosure is defined not by the detailed description, but by the claims and their equivalents, and all variations within the scope of the claims and their equivalents are to be construed as being included in the disclosure.

What is claimed is:

1. A method for diagnosing cancer, the method comprising:
(a) measuring an expression level of a KCa3.1 channel protein or the mRNA expressed from a gene encoding the KCa3.1 channel protein (i) in the red blood cells of a subject suspected of having cancer, or (ii) in vascular endothelial cells exposed to the serum of a subject suspected of having cancer;
(b) comparing the expression level of the protein or mRNA measured in the step (a) with an expression level measured in a normal control sample;
(c) measuring an expression level of at least one protein selected from the group consisting of clathrin, caveolin1, EEA and Rab5C, or the mRNA expressed from a gene encoding the at least one protein of the subject in the step (a); and
(d) comparing the expression level of the protein or mRNA measured in the step (c) with an expression level measured in a normal control sample.

2. The method of claim 1, further comprising:
(e) calculating a ratio of the expression level measured in the step (a) and the expression level measured in the step (c), and
(f) comparing the ratio calculated in step (e) with a ratio of the expression levels measured in the normal control sample.

3. The method of claim 1, wherein the KCa 3.1 channel protein expression level is measured using an antibody or an aptamer capable specifically binding to the KCa 3.1 channel protein.

4. The method of claim 1, wherein the mRNA expression levels in steps (a) and (c) are measured by a primer, a probe, or an antisense oligonucleotide capable of specifically binding to the gene.

5. The method of claim 1, wherein the cancer is one selected from the group consisting of liver cancer, lung cancer, gastric cancer, pancreatic cancer, renal cell carcinoma, uterine cancer, cervical cancer, brain cancer, oral cancer, colon cancer, biliary cancer, bone cancer, and skin cancer.
